(19) United States
(12) Patent Application Publication
(10) Pub. No.: US 2012/0101454 A1
(43) Pub. Date: Apr. 26, 2012

(54) SPLASH FREE NEEDLE ASSEMBLY

(76) Inventor: Gloria Lovell, Altadena, CA (US)

(21) Appl. No.: 12/910,755

(22) Filed: Oct. 22, 2010

(51) Int. Cl. A61M 25/14

(52) U.S. Cl. 604/247

(57) ABSTRACT

A leak preventing AV needle assembly including an AV needle, a catheter having a first end engaged with the AV needle and a second end, and a one-way valve assembly including a housing having a first end portion engaged with the second end of the catheter, and a second end portion having an opening for receiving a fluid transmission conduit, is disclosed. The valve assembly further includes an axially displaceable closure element for closing the opening in the second end portion when the second end portion is not engaged with the conduit. The needle assembly further includes an elongated housing within which is disposed a biasing element for urging the closure element away from the first end and into sealing engagement with an opening in the second end of the valve assembly housing into which a fluid transmission conduit can be inserted for fluid communication with a catheter inserted into the first end of the valve housing. The closure element can be a disk, an element with a spherical portion, or an element with a conical portion.

### TECHNICAL FIELD

The present invention relates generally to medical insertion assemblies, such as a needle or a catheter, and more particularly to an insertion assembly, including a needle having a connector on one end for safely and securely connecting to and disconnecting from a length of a fluid conveying conduit leading to or away from a fluid receptacle, the connector including a valve to prevent leakage from the needle or catheter when either is disconnected from the fluid conveying conduit.

### BACKGROUND OF THE INVENTION

Intravenous (IV) therapy is a versatile technique used for the administration and/or removal of medical fluids to and from patients. IV therapy has been used for various purposes such as the maintenance of fluid and electrolyte balance, the transfusion of blood, the administration of nutritional supplements, chemotherapy, the administration of drugs and medications, and the draining of selected bodily fluids. Fluids may be administered intravenously by injection through a hypodermic syringe, or intermittently or continuously by infusion using a needle or a plastic or silicone catheter.

Although there are many advantages to be derived by the patient from the use of IV techniques, the past two decades have brought heightened awareness of the risks of propagating infectious diseases associated with these procedures, particularly due to the HIV virus. One method by which infectious disease may be spread is an inadvertent puncture of medical personnel by the sharp needle that was used to insert an IV needle or catheter into an infected patient. The insertion ends of IV needles or catheters are extremely sharp and should the insertion site become wet with administration fluid or blood, the medical personnel may inadvertently puncture herself or himself while handling the sharp needle, or already have an opening in the skin, which raises the possibility of contracting an infection or worse.

One consequence of this heightened awareness has been the development of various devices designed to reduce the risk of spreading infectious diseases during the IV procedure. Devices have been developed over the years to attempt to lower the risk of such inadvertent punctures. One such class of devices has been the blunt cannula approach to connectors. In this class of connectors, sharp needles are not used. In one approach in this class, the connecting devices have been designed based on the Luer system of connectors in which the mating surfaces are tapered. While this approach has advanced the safety of health care workers substantially, a venipuncture procedure still requires the use of a sharp needle.

Another class of devices developed in the venipuncture area is the safety needle. This is a type of needle housed inside a protective guard. It comprises a sharp needle that may be extended from and retracted into the protective guard, the purpose of which is to lessen the chance of an inadvertent needle puncture of medical personnel. It provides the required sharp needle for the venipuncture procedure but carefully guards the operator against the sharp needle except for the one time it is extended to pierce the patient. In some versions, the sharp needle is locked within the protective guard once it has been retracted. The possibility of an inadvertent puncture is further reduced with such an arrangement.

Catheters used for peripheral IV therapy tend to be relatively short, although they are occasionally of greater length for insertion into a deep vein. A peripheral IV catheter is made of soft, flexible plastic or silicone. In the conventional venipuncture procedure, the catheter is inserted into a vein in the patient's hand, foot, or the inner aspect of the arm or any vein in the body that will accept an IV catheter.

IV catheters are used to provide fluid to or to withdraw fluid from a patient. In order to properly place an IV catheter in a patient's vein, a sharp needle or similar element must be used to puncture the skin, tissue, and vein wall to provide a path for placement of the catheter in the vein. Typical IV catheters are “over-the needle” catheters where the catheter is coaxially placed over the needle. The catheter thus rides with the needle through the skin, tissue, and vein wall and into the patient's vein. When the needle pierces the vein, blood will “flashback” into the needle. Thus, once the clinician observes this flashback of blood, the clinician will know that the catheter and needle have been inserted in the vein. The needle can then be withdrawn from the patient and the catheter can be advanced further into the vein as desired.

During the above venipuncture procedure, medical personnel are exposed to the possibility of an accidental needle puncture or to contamination from the back flow of the patient's blood from the venipuncture site. While care is usually taken to avoid a puncture of the medical personnel, circumstances may arise during which a puncture of the medical personnel may nevertheless occur exposing the health care personnel to any contaminated fluids, including infectious blood borne diseases, emanating from the patient.

There is therefore a great need for apparatus incorporated in IV needles or catheters that will prevent medical personnel who handle such IV paraphernalia from exposure to bodily fluids.

### SUMMARY OF THE INVENTION

One aspect of the present invention is to provide a leakage prevention mechanism for incorporation in an IV needle or an arteriovenous (AV) catheter for preventing medical or bodily fluids, such as blood, plasma, medication solutions, urine, and the like, in the needle or catheter from draining or leaking out when the needle or catheter is disconnected from a conduit for the medical or bodily fluids.

Another aspect of the invention is to provide a one-way valve at the end of an IV needle or AV catheter that is connectable to a fluid conduit for delivering or receiving medical fluids thereto or therefrom, in which the one-way valve includes a closure element engagable in an opening of the housing and a biasing element positioned in the housing and urging the closure element into sealing engagement with the opening so that fluids in the housing cannot leak out.

### BRIEF DESCRIPTION OF THE DRAWINGS

FIG. 1 is a schematic illustration of an AV needle assembly having one-way valve combined with a catheter, according to the present invention; and

FIG. 2 is a cross-sectional view of the one-way valve contemplated by the present invention.

### DETAILED DESCRIPTION OF THE INVENTION

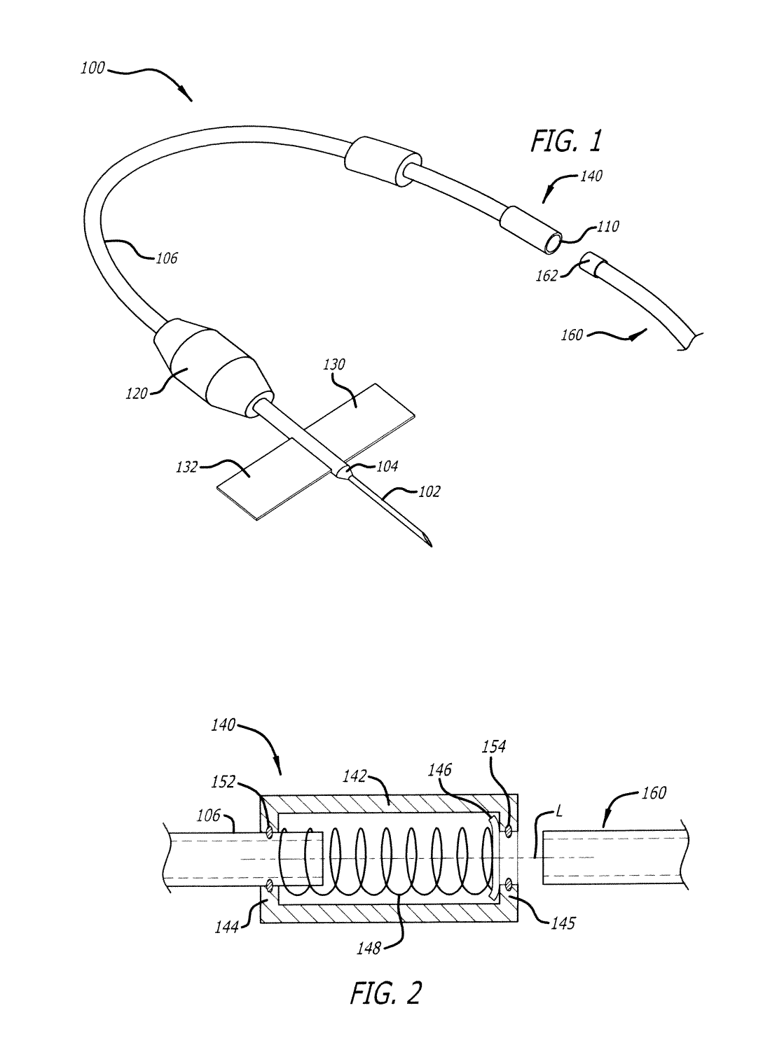

Referring first to FIG. 1, there is shown an arteriovenous (AV) needle assembly 100 including a needle 102, a needle holder 104, and a catheter 106 extending rearwardly therefrom terminating in an end connector portion 110. A fluid conduit 160, leading to or from a medical fluid receptacle (not shown), carries on one end a connector 162 configured for mating connection with end connector portion 110. A valve assembly, shown generally at 140, is housed within the end connection portion 110. The needle assembly may include a protective guard element 120 to cover the portion of the catheter adjacent the needle 102, which serves the purpose of insuring that the catheter is not inadvertently knocked or dislodged from the rear end of the needle holder 104 or from the needle insertion site on the patient. A butterfly element 130, having opposed wing elements 132, 134 is disposed on opposite sides of the needle holder to secure the needle and needle holder to the patient at the needle insertion site. Each of the wing elements 132, 134 may have a surface bearing an adhesive for engagement with the patient's skin in close proximity to the needle insertion site.

The present invention also encompasses the use of a catheter, without the needle and needle holder, where the catheter includes a front end with a beveled nose portion configured for insertion into an insertion site in the patient's body, and a rear end provided with connector element 110 for making connection with a fluid conduit 160 used to deliver fluid to or from the patient. The rear end of the catheter includes a valve assembly 140 located at the end of the catheter, in the same manner as described above and as follows.

FIG. 2 is an example of a valve assembly 140 that can be employed in the assembly of the present invention. As shown, the valve assembly includes a housing 142 having a longitudinal axis “L” and opposing end walls 144, 145, each having a cylindrical opening of a diameter less than the diameter of the end wall in which the opening is located. Each respective opening engages a conduit element, as for example the cylindrical needle holder 104 or the catheter 106. A closure element 146 is disposed within the housing adjacent one end wall 145, and is normally urged, by a resilient member 148, into sealing engagement with the opening in that end. The resilient member 148 can be a coiled spring element, a compressible rubber or plastic element, or a gas-filled bladder element. The closure element 146, which can be configured in a variety of shapes, as for example a thin disk, a cone shaped element, a ball mounted to a disk shaped platform, and the like, will be pushed toward and into the opening so that any fluid in the housing that remains after the fluid conduit 160 is removed from the housing, is prevented from dripping or leaking out. Annular seals 152 are mounted in grooves 154 in the annular surfaces of the end wall openings to insure a fluid-tight connection between the fluid conveying conduits 106 and 160, on the one hand, and the end walls of the housing 142, on the other hand.

When the fluid conduit 160 is inserted into the housing 142 through the opening in the end wall 145, the free “insertion” end 162 of the conduit 160 engages the closure element 146 and pushes it away from the opening into the housing against the resilient member 148, and in so doing creates an unobstructed flow path from the conduit 160 through the housing interior and into fluid communication with the catheter 106. When the free end 162 of the fluid conduit 160 is withdrawn from the opening 146 in the end wall 145, the resilient member 148 urges the closure element 146 back into sealing engagement with the opening in the end wall 145.

Those skilled in the art will appreciate that various adoptions and modifications of the invention as described above can be configured without departing from the scope and spirit of the invention. Therefore, it is to be understood that, within the scope of the appended claims, the invention may be practiced other than as specifically described herein.

What is claimed is:

1. A leak preventing AV needle assembly, comprising:
an AV needle;
a catheter having a first end engaged with the AV needle, and a second end; and
a one-way valve assembly comprising a housing having a first end portion engaged with the second end of the catheter, and a second end portion having an opening for receiving a fluid transmission conduit;
the valve assembly further including an axially displaceable closure element for closing the opening in the second end portion when the second end portion is not engaged with the conduit.

2. The leak preventing AV needle assembly of claim 1 further including a biasing element disposed within the valve assembly and arranged between the first end and the closure element for urging the closure element into sealing engagement with the opening in the second end of the valve assembly housing.

3. The leak preventing AV needle assembly of claim 1, wherein the housing is elongated with a longitudinal axis, the first end portion of the housing defining a cylindrical wall normal to the longitudinal axis and having an opening for secure engagement with the catheter, and the second end portion of the housing defining a cylindrical wall normal to the longitudinal axis of the housing and having an opening for secure engagement with a fluid transmission conduit, wherein the closure element is configured to block the opening of the housing second end portion; and
a biasing element urging the closure element toward the second end portion of the housing.

4. The leak preventing AV needle assembly of claim 3, wherein the closure element comprises a disk.

5. The leak preventing AV needle assembly of claim 3, wherein the closure element comprises a spherical portion.

6. The leak preventing AV needle assembly of claim 3, wherein the closure element comprises a conical portion.

7. In combination with one end of a medical fluid conveyance attached between an AV needle and a fluid transmission conduit, a one-way valve comprising:
a cylindrical chamber having one end engagable with the catheter and the other end engageable with the fluid transmission conduit;
a closure element arranged inside the chamber and positioned adjacent the other end of the chamber, the outer diameter of the closure element being substantially equal to the inner diameter of the chamber; and
a spring element positioned between the closure element and the one end of the chamber, the spring element normally biasing the closure element into engagement with the other end of the chamber.

8. The one-way valve of claim 7, wherein the closure element comprises a disk.

9. The one-way valve of claim 7, wherein the closure element comprises a spherical portion.

10. The one-way valve of claim 7, wherein the closure element comprises a conical portion.
